(19) United States
(12) Patent Application Publication
(10) Pub. No.: US 2008/0003296 A1
(43) Pub. Date: Jan. 3, 2008

(54) Amorphous Cyclodextrin Compositions

(76) Inventor: Rodney James Ketner, Bend, OR (US)

Correspondence Address: CHERNOFF, VILHAUER, MCCLUNG & STENZEL, 1600 ODS TOWER, 601 SW SECOND AVENUE, PORTLAND, OR, 97204-3157, US

(21) Appl. No.: 11/579,191

(22) PCT Filed: Jul. 11, 2005
(86) PCT No.: PCT/IB05/02190

§ 371 Date: Oct. 30, 2006

(51) Int. Cl. A61K 47/48; A61K 47/40

(52) U.S. Cl. 424/499; 514/772.3

(57) ABSTRACT

Compositions are disclosed that comprise a plurality of particles of a cyclodextrin and a water-soluble polymer wherein the cyclodextrin is in intimate contact with the polymer and a major portion of the cyclodextrin in the particles is amorphous.

### BACKGROUND OF THE INVENTION

The present invention relates to compositions comprising amorphous cyclodextrin and a water-soluble polymer.

Cyclodextrins are cyclic multicyclopyranose units connected by alpha-(1,4) linkages. The most widely known cyclodextrins are alpha, beta and gamma-cyclodextrins. Derivatives of these cyclodextrins are also known and used in the pharmaceutical field. The cyclic nature of the cyclodextrins, the hydrophobic properties of their cavities as well as the hydrophilic character of their outer surfaces, enables them to interact with other chemicals and to produce inclusion compounds.

Numerous reviews and patents related to the use of cyclodextrins and their derivatives to prepare inclusion complexes of active agents are found in the literature, for example, D. Duchene, Cyclodextrins and their Industrial Uses, Editions de Sante, Paris, 1987, Chapter 6 (211-257), Chapter 8 (297-350), Chapter 10 (393-439); D. Duchene et al, Acta Pharma Technol. 36(1)6, 1-6,1990; D. Duchene et al, Drug Dev. Ind. Pharm., 16(17), 2487-2499, 1990; C. Hunter et al, European Patent Publication No. EP 0346006, December 1988. See also U.S. Pat. Nos. 4,024,223; 4,228,160; 4,232,009; 4,351,846; 4,352,793; 4,383,992; 4,407,795; 4,424,209; 4,425,336; 4,438,106; 4,474,811; 4,478,995; 4,479,944; 4,479,966; 4,497,803; 4,499,085; 4,524,068; 4,555,504; 4,565,807; 4,575,548; 4,598,070; 4,603,123; 4,608,366; 4,623,641; 4,659,696; 4,663,316; 4,675,395; 4,728,509; 4,728,510; and 4,751,095.

Inclusion complexes prepared to specifically improve water solubility and hence bioavailability of poorly soluble drugs have been reported by workers such as D. D. Chow et al, Int. J. Pharm., 28, 95-101, 1986; F. A. Menard et al, Drug Dev. Ind. Pharm., 14(11), 1529-1547, 1988; F. J. Otera-Espinar et al, Int. J. Pharm., 75, 37-44, 1991; and Berand M. Markarian et al, European Patent Publication No. EP 0274444, July 1988. Chemical modifications of cyclodextrins to prepare derivatives that further improve solubility of water insoluble drugs have been described, for example, by J. Pitha, U.S. Pat. No. 4,727,064, February 1988; N. S. Bodor, U.S. Pat. No. 5,024,998, July 1991.

It is known that some types of cyclodextrins are capable of crystallizing in vivo, significantly limiting the effectiveness of cyclodextrins for solubilizing poorly soluble drugs. To overcome this limitation, researchers have chemically modified the cyclodextrins to prevent crystallization. See for example, U.S. Patent Application Publication No. 2003-0148996A1, U.S. Pat. Nos. 6,407,061, 6,407,061, 6,342,478, 6,313,093, 6,180,603, 5,624,898, 5,296,472, 5,180,716, 4,596,795, J. Pitha, J. Contr. Rel., 6:309-313 (1987), J. Pitha et al., Life Sciences, 43:493-502 (1988), T. Irie, Pharm. Res., 5(11):713-717 (1988), J. Pitha, Neurotransmissions, V(1):1-4 (1989). These chemically modified cyclodextrins are typically a mixture of modified products with varying degrees and types of substituents, which prevents crystallization.

Nevertheless, there is still a need to provide amorphous forms of cyclodextrin that do not require chemical modification of the cyclodextrin, and that overcomes other drawbacks of the prior art. These needs are met by the present invention, which is summarized and described in detail below.

### BRIEF SUMMARY OF THE INVENTION

In a first aspect, a composition comprises a solid composition comprising a plurality of particles. Each of the particles comprises a cyclodextrin and a water-soluble polymer. The cyclodextrin is in intimate contact with the water-soluble polymer in the particles. At least a major portion of the cyclodextrin in the particles is amorphous.

In a second aspect, a composition comprises an active and a plurality of particles, each of the particles comprising a cyclodextrin and a water-soluble polymer, wherein the cyclodextrin is in intimate contact with the water-soluble polymer, and wherein at least a major portion of the cyclodextrin is amorphous.

In a third aspect, a process is provided for making a plurality of particles, each of the particles comprising a cyclodextrin and a water-soluble polymer, the process comprising: (a) forming a solution comprising cyclodextrin, a water-soluble polymer, and a solvent, (b) rapidly removing the solvent from the solution to form a solid, and (c) forming particles from the so-formed solid, wherein at least a major portion of the cyclodextrin is amorphous. In one embodiment, steps (b) and (c) are preformed simultaneously by spray-drying the solution into a chamber.

The present invention overcomes the drawbacks of the prior art in that the cyclodextrin is present in an amorphous form without needing to chemically modify the cyclodextrin. The water-soluble polymer is present in a sufficient amount to retard crystallization of the cyclodextrin in the particles, effectively stabilizing the amorphous form of the cyclodextrin. The compositions of the present invention comprising amorphous cyclodextrins have an advantage of resulting in faster dissolution and/or dispersing of the cyclodextrin when administered to an aqueous use environment. This can lead to more rapid formation of inclusion complexes with actives. When the active has a low aqueous solubility, this more rapid formation of an inclusion complex can lead to improved solubilization of the active in the aqueous use environment.

As used herein, an “active” is meant a compound that can form an inclusion complex or otherwise associate with a cyclodextrin. Examples of such materials include pharmaceuticals, vitamins, nutriceuticals, agrochemical compounds, nutrients, fertilizers, pesticides, fungicides, botanical extracts, flavoring agents, fruit extracts, spices, cosmetics, coloring agents, pigments, and the like.

As used herein, an “aqueous use environment” refers to any environment that contains water in which it may be desirable to deliver, use, or otherwise contain the active. For example, when the active is a pharmaceutical, the aqueous use environment may be in vivo fluids, such as present in the buccal space or the GI tract of an animal, such as a mammal, and particularly a human. When the active is an agrochemical compound, the aqueous use environment may be the mass of the vegetation or the soil in which the vegetation is planted. Alternatively, the aqueous use environment may be an in vitro environment of a test solution, such as unbuffered water, a simulated mouth buffer (MB) or a simulated gastric buffer (GB). An appropriate simulated MB test solution is 0.05M KH₂PO₄ buffer adjusted to pH 7.3 with 10M KOH. Appropriate GB test solutions include 0.01N HCl and 0.1N HCl. “Administration” to a use environment means, where the in vivo use environment is the mouth or GI tract, ingestion or other such means to deliver the composition. Where the use environment is in vitro, “administration” refers to placement or delivery of the composition or dosage form containing the composition to the in vitro test medium.

The foregoing and other objectives, features, and advantages of the invention will be more readily understood upon consideration of the following detailed description of the invention.

### BRIEF DESCRIPTION OF THE SEVERAL VIEWS OF THE DRAWINGS

FIG. 1 is an X-ray diffraction pattern of the crystalline β-cyclodextrin used to form the particles in Example 1.

FIG. 2 is an X-ray diffraction pattern of particles of β-cyclodextrin and HPMC formed in Example 1 showing no crystalline cyclodextrin in the particles.

### DETAILED DESCRIPTION OF PREFERRED EMBODIMENTS

The present invention relates to solid compositions of a cyclodextrin and a water-soluble polymer. At least a major portion of the cyclodextrin in the particles is amorphous. The nature of the solid compositions, suitable cyclodextrins and water-soluble polymers, and methods for making the compositions are discussed in more detail below.

### Cyclodextrins

The solid compositions of the present invention comprise a cyclodextrin. Cyclodextrins useful in the present invention include α-, β- and γ-cyclodextrins and alkyl and hydroxyalkyl derivatives thereof, with β-cyclodextrins and derivatives of β-cyclodextrin being the most preferred from the standpoint of availability and cost. Exemplary derivatives of cyclodextrin include mono- or polyalkylated β-cyclodextrin, mono- or polyhydroxyalkylated β-cyclodextrin, mono, tetra or hepta-substituted β-cyclodextrin, and sulfoalkyl ether cyclodextrin (SAE-CD). Specific cyclodextrin derivatives for use herein include hydroxypropyl-β-cyclodextrin, hydroxyethyl-β-cyclodextrin, hydroxypropyl-γ-cyclodextrin, hydroxyethyl-γ-cyclodextrin, dihydroxypropyl-β-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-β-cyclodextrin, maltotriosyl-β-cyclodextrin, maltotriosyl-γ-cyclodextrin, dimaltosyl-β-cyclodextrin, methyl-β-cyclodextrin, and mixtures thereof such as maltosyl-β-cyclodextrin/dimaltosyl-β-cyclodextrin. A preferred cyclodextrin is β-cyclodextrin.

### Water-Soluble Polymers

The solid compositions of the present invention also comprise a water-soluble polymer. As used herein, the term “polymer,” which can be a mixture of polymers, is used conventionally, meaning a compound that is made of monomers connected together to form a larger molecule. A polymeric component generally consists of at least about 20 monomers. Thus, the molecular weight of a polymeric component will generally be about 2000 daltons or more. The term “water-soluble” means the polymer has an aqueous solubility of at least about 0.1 mg/mL over at least a portion of the pH range of 1 to 8. Preferably, the polymer has an aqueous solubility of at least about 0.5 mg/mL, and more preferably at least about 1 mg/mL.

In general, polymers useful in the compositions of the present invention may be neutral or ionizable cellulosic or non-cellulosic polymers. Examples of neutral non-cellulosic polymers include vinyl polymers and copolymers, polyvinyl alcohols, polyvinyl alcohol/polyvinyl acetate copolymers, polyvinyl pyrrolidone polyethylene glycol/polypropylene glycol copolymers, polyvinyl pyrrolidone, polyethylene/polyvinyl alcohol copolymers, and polyoxyethylene/polyoxypropylene block copolymers (also known as poloxamers). Examples of ionizable non-cellulosic polymers include carboxylic acid functionalized polymethacrylates and carboxylic acid functionalized polyacrylates, amine-functionalized polyacrylates and polymethacrylates, high molecular weight proteins such as gelatin and albumin, and carboxylic acid functionalized starches such as starch glycolate. Examples of neutral cellulosic polymers are hydroxypropyl methyl cellulose (HPMC), hydroxyethyl cellulose (HEC), hydroxypropyl cellulose (HPC), methyl cellulose, hydroxyethyl methyl cellulose, and hydroxyethyl ethyl cellulose. Examples of ionizable cellulosic polymers are hydroxypropyl methyl cellulose acetate succinate (HPMCAS), hydroxypropyl methyl cellulose phthalate (HPMCP), carboxymethyl ethyl cellulose (CMEC), carboxyethyl cellulose, carboxymethyl cellulose, cellulose acetate phthalate, hydroxypropyl methyl cellulose acetate phthalate, and cellulose acetate trimellitate. Neutralized forms of ionizable polymers may also be useful, such as sodium carboxymethyl cellulose (sodium CMC). Preferred water-soluble polymers include polyvinyl alcohols, polyvinyl pyrrolidone, poloxamers, polymethacrylates and polyacrylates, gelatin, HPMC, HEC, HPC, HPMCAS, HPMCP, CMEC, and sodium CMC.

### Solid Particles of Cyclodextrin and Polymer

The compositions of the present invention comprise a plurality of particles. Each of these particles comprises both a cyclodextrin and a water-soluble polymer. The amount of polymer to the amount of cyclodextrin in each of the particles depends on the characteristics of the polymer and cyclodextrin and may vary widely from a cyclodextrin-to-polymer weight ratio of from about 0.01 (1 part cyclodextrin to 100 parts polymer) to about 100 (e.g., 1 wt % cyclodextrin to 99 wt % cyclodextrin). In order to keep the total mass of the composition small, it is preferred that the particles comprise higher amounts of cyclodextrin. Thus, the cyclodextrin-to-polymer weight ratio preferably is at least about 0.05 (about 5 wt % cyclodextrin), more preferably at least about 0.1 (about 10 wt % cyclodextrin), even more preferably at least about 0.2 (about 17 wt % cyclodextrin), and even more preferably at least about 0.33 (about 25 wt % cyclodextrin).

The particles should also contain a sufficient amount of water-soluble polymer to stabilize the amorphous form of the cyclodextrin in the particles, retarding crystallization of the cyclodextrin. Thus, the cyclodextrin-to-polymer weight ratio is preferably less than about 49 (about 98 wt % cyclodextrin), more preferably less than about 19 (about 95 wt % cyclodextrin), more preferably less than about 9 (about 90 wt % cyclodextrin), and even more preferably less than about 3 (about 75 wt % cyclodextrin).

At least a major portion of the cyclodextrin in the particles is amorphous. By “amorphous” is meant that the cyclodextrin is in a non-crystalline state. As used herein, the term “a major portion” of the cyclodextrin means that at least 60 wt % of the cyclodextrin in the particles is in the amorphous form, rather than the crystalline form. Preferably “a substantial portion” of the cyclodextrin in the particles is amorphous, meaning that at least 75 wt % of the cyclodextrin in the particles is in the amorphous form. More preferably the cyclodextrin is “almost completely amorphous,” meaning that the amount of drug in the crystalline form does not exceed 10 wt %. Amounts of crystalline cyclodextrin may be measured by powder X-ray diffraction, Scanning Electron Microscope (SEM) analysis, differential scanning calorimetry (DSC), or any other standard quantitative measurement. Most preferably the particles are substantially free of crystalline cyclodextrin.

The amorphous cyclodextrin in the particles is in intimate contact with the water-soluble polymer. The amorphous cyclodextrin in the particle can exist as a pure phase, as a solid solution of cyclodextrin homogeneously distributed throughout the water-soluble polymer, or any combination of these states or those states that lie intermediate between them. In cases where cyclodextrin-rich amorphous domains exist, these domains are generally quite small; that is, less than about 1 μm in size. Preferably, such domains are less than about 100 nm in size. The particles may have a single glass-transition temperature, indicating that the cyclodextrin is homogeneously dispersed throughout the water-soluble polymer, or may have two glass-transition temperatures, corresponding to a cyclodextrin-rich amorphous phase and a cyclodextrin-poor amorphous phase.

The primary constituents of the particles are the cyclodextrin and the water-soluble polymer. The cyclodextrin and water-soluble polymer together constitute at least 50 wt % of the particles. The cyclodextrin and water-soluble polymer may constitute even greater amounts of the composition, and may constitute at least 60 wt %, at least 70 wt %, at least 80 wt %, or even at least 90 wt % of the particles. In one embodiment, the particles consist essentially of the cyclodextrin and water-soluble polymer.

### Methods for Making Particles of Cyclodextrin and Polymer

The particles of cyclodextrin and water-soluble polymer of the present invention may be made according to any known process that results in at least a major portion (that is, at least 60 wt %) of the cyclodextrin being in the amorphous state. One preferred technique is solvent processing. In solvent processing, the cyclodextrin and water-soluble polymer are dissolved in a common solvent and the solvent subsequently removed by evaporation or by mixing with a non-solvent. “Common” here means that the solvent, which can be a mixture of compounds, will dissolve both the cyclodextrin and the polymer. After both the cyclodextrin and the polymer have been dissolved, the solvent is removed by evaporation or by mixing with a non-solvent. Exemplary processes are spray-drying, spray-coating (pan-coating, fluidized bed coating, etc.), evaporation, lyophilization, and precipitation by rapid mixing of the polymer and cyclodextrin solution with CO₂, or some other non-solvent.

Solvents suitable for solvent processing are preferably volatile with a boiling point of 150° C. or less. In addition, the solvent should have relatively low toxicity and be removed from the particles to a level that is acceptable according to The International Committee on Harmonization (ICH) guidelines. Removal of solvent to this level may require a subsequent processing step such as tray drying. Preferred solvents include water; alcohols such as methanol, and ethanol; ketones such as acetone, methyl ethyl ketone and methyl iso-butyl ketone; and various other solvents such as acetonitrile, methylene chloride, and tetrahydrofuran. Lower volatility solvents such as dimethyl acetamide or dimethylsulfoxide can also be used in small amounts in mixtures with a volatile solvent. Mixtures of solvents, such as 50% methanol and 50% acetone, can also be used, as can mixtures with water, so long as the polymer and cyclodextrin are sufficiently soluble to make the process practicable.

The solvent may be removed by spray-drying. The term “spray-drying” is used conventionally and broadly refers to processes involving breaking up liquid mixtures into small droplets (atomization) and rapidly removing solvent from the mixture in a spray-drying apparatus where there is a strong driving force for evaporation of solvent from the droplets. Spray-drying processes and spray-drying equipment are described generally in Perry's Chemical Engineers' Handbook, pages 20-54 to 20-57 (Sixth Edition 1984). More details on spray-drying processes and equipment are reviewed by Marshall, “Atomization and Spray-Drying,” 50 Chem. Eng. Prog. Monogr. Series 2 (1954), and Masters, Spray Drying Handbook (Fourth Edition 1985). The strong driving force for solvent evaporation is generally provided by maintaining the partial pressure of solvent in the spray-drying apparatus well below the vapor pressure of the solvent at the temperature of the drying droplets. This is accomplished by (1) maintaining the pressure in the spray-drying apparatus at a partial vacuum (e.g., 0.01 to 0.50 atm); or (2) mixing the liquid droplets with a warm drying gas; or (3) both (1) and (2). In addition, at least a portion of the heat required for evaporation of solvent may be provided by heating the spray solution.

The solvent-bearing feed, comprising the cyclodextrin and the water-soluble polymer, can be spray-dried under a wide variety of conditions and yet still yield particles with acceptable properties. For example, various types of nozzles can be used to atomize the spray solution, thereby introducing the spray solution into the spray-dry chamber as a collection of small droplets. Essentially any type of nozzle may be used to spray the solution as long as the droplets that are formed are sufficiently small that they dry sufficiently (due to evaporation of solvent) that they do not stick to or coat the spray-drying chamber wall.

Although the maximum droplet size varies widely as a function of the size, shape and flow pattern within the spray-dryer, generally droplets should be less than about 500 μm in diameter when they exit the nozzle. Examples of types of nozzles that may be used to form the solid particles include the two-fluid nozzle, the fountain-type nozzle, the flat fan-type nozzle, the pressure nozzle and the rotary atomizer.

The spray solution can be delivered to the spray nozzle or nozzles at a wide range of temperatures and flow rates. Generally, the spray solution temperature can range anywhere from just above the solvent's freezing point to about 20° C. above its ambient pressure boiling point (by pressurizing the solution) and in some cases even higher. Spray solution flow rates to the spray nozzle can vary over a wide range depending on the type of nozzle, spray-dryer size and spray-dry conditions such as the inlet temperature and flow rate of the drying gas. Generally, the energy for evaporation of solvent from the spray solution in a spray-drying process comes primarily from the drying gas.

The drying gas can, in principle, be essentially any gas, but for safety reasons and to minimize undesirable oxidation of the cyclodextrin or other materials in the solid composition, an inert gas such as nitrogen, nitrogen-enriched air or argon is utilized. The drying gas is typically introduced into the drying chamber at a temperature between about 60° and about 300° C. and preferably between about 80° and about 240° C.

The large surface-to-volume ratio of the droplets and the large driving force for evaporation of solvent leads to rapid solidification times for the droplets. Solidification times should be less than about 20 seconds, preferably less than about 10 seconds, and more preferably less than 1 second. This rapid solidification is often critical to the particles maintaining a major portion of amorphous cyclodextrin.

Following solidification, the solid powder typically stays in the spray-drying chamber for about 5 to 60 seconds, further evaporating solvent from the solid powder. The final solvent content of the particles as they exit the dryer should be low, since this reduces the mobility of the cyclodextrin molecules in the particles, thereby improving its stability. Generally, the solvent content of the particles as they leave the spray-drying chamber should be less than 10 wt % and preferably less than 2 wt %. Following formation, the particles can be dried to remove residual solvent using suitable drying processes, such as tray drying, fluid bed drying, microwave drying, belt drying, rotary drying, vacuum drying, and other drying processes known in the art.

In another embodiment, the particles are formed by an evaporation process. In this process the cyclodextrin and water-soluble polymer are dissolved in a common solvent as described above. The solvent is then removed by evaporation to form the solid composition. Examples of evaporation processes include rotoevaporation, multiple-effect evaporation, flash evaporation, and film evaporation. The resulting solids are preferably formed into small particles, such as by using a mortar and pestle or other milling processes known in the art. The particles may be sieved and dried as necessary to obtain a material with the desired properties.

In another embodiment, the particles are formed by spraying a coating solution of the cyclodextrin and water-soluble polymer onto seed cores. The seed cores can be made from any suitable material such as starch, microcrystalline cellulose, sugar or wax, by any known method, such as melt- or spray-congealing, extrusion/spheronization, granulation, spray-drying and the like.

The coating solution can be sprayed onto such seed cores using coating equipment known in the pharmaceutical arts, such as pan coaters (e.g., Hi-Coater available from Freund Corp. of Tokyo, Japan, Accela-Cota available from Manesty of Liverpool, U.K.), fluidized bed coaters (e.g., Wurster coaters or top-sprayers available from Glatt Air Technologies of Ramsey, N.J. and from Niro Pharma Systems of Bubendorf, Switzerland) and rotary granulators (e.g., CF-Granulator, available from Freund Corp).

The particles may also be made by a lyophilization process. In lyophilization processes, also known in the art as freeze-drying processes, a solution of the cyclodextrin and polymer in a solvent is frozen into a solid state. The solvent is then removed from the solid by sublimation using a vacuum. After the solvent has been removed, the solids are preferably formed into small particles, such as by using a mortar and pestle or other milling processes known in the art.

Once the particles comprising the cyclodextrin and water-soluble polymer have been formed, several processing operations can be used to facilitate incorporation of the particles into compositions. These processing operations include drying, granulation, and milling.

The particles may be granulated to increase their size and improve handling of the particles while forming a suitable composition. Preferably, the average size of the granules will range from 50 to 1000 μm. Dry or wet granulation processes can be used for this purpose. An example of a dry granulation process is roller compaction. Wet granulation processes can include so-called low shear and high shear granulation, as well as fluid bed granulation. In these processes, a granulation fluid is mixed with the composition after the dry components have been blended to aid in the formation of the granulated composition. Examples of granulation fluids include water, ethanol, isopropyl alcohol, n-propanol, the various isomers of butanol, and mixtures thereof. A polymer may be added with the granulation fluid to aid in granulating the particles. Examples of suitable polymers include poloxamers, hydroxypropyl cellulose, hydroxyethyl cellulose, and hydroxypropyl methylcellulose.

If a wet granulation process is used, the granulated composition is often dried prior to further processing. Examples of suitable drying processes to be used in connection with wet granulation are the same as those described above. Where the particles are made by a solvent process, the composition can be granulated prior to removal of residual solvent. During the drying process, residual solvent and granulation fluid are concurrently removed from the composition.

Once the particles have been granulated, they may then be milled to achieve the desired particle size. Examples of suitable processes for milling the granules include hammer milling, ball milling, fluid-energy milling, roller milling, cutting milling, and other milling processes known in the art.

### Actives

In one embodiment, a composition of the present invention comprises an active. The active can be any compound that can form an inclusion complex or otherwise associate with a cyclodextrin. Examples of suitable actives include pharmaceuticals, vitamins, nutriceuticals, agrochemical compounds, nutrients, fertilizers, pesticides, fungicides, botanical extracts, flavoring agents, fruit extracts, spices, cosmetics, coloring agents, pigments, and the like.

One particular class of actives that may benefit from the compositions of the present invention is pharmaceuticals, and in particular, unpleasant tasting drugs. The term “drug” is conventional, denoting a compound having beneficial prophylactic and/or therapeutic properties when administered to an animal, especially humans. Cyclodextrins can be used to taste mask unpleasant tasting drugs. Cyclodextrins form complexes with the drug in an aqueous environment that prevents contact of drug with the taste buds; often these complexes have improved taste over the uncomplexed drug. The compositions of the present invention comprising particles of amorphous cyclodextrin and a water-soluble polymer can be mixed with unpleasant tasting drugs to effectively taste-mask the drug. Tastemasking unpleasant drugs with the compositions of the present invention are disclosed in detail in co-pending U.S. patent application Ser. No. ______ (Attorney Docket No. PC25840), incorporated herein by reference.

Exemplary drugs that may be used with the current invention include, without limitation, inorganic and organic compounds that act on the peripheral nerves, adrenergic receptors, cholinergic receptors, nervous system, skeletal muscles, cardiovascular smooth muscles, blood circulatory system, synaptic sites, neuroeffector junctional sites, endocrine and hormone systems, immunological system, reproductive system, autocoid systems, alimentary and excretary systems, inhibitors of autocoids and histamine systems. Preferred classes of drugs include, but are not limited to, antacids, analgesics, anti-anginals, anti-anxiety agents, anti-arrhythmics, anti-bacterials, antibiotics, anti-diarrheals, anti-depressants, anti-epileptics, anti-fungals, anti-histamines, anti-hypertensives, anti-inflammatory agents, anti-virals, cardiac agents, contraceptives, cough suppressants, cytotoxics, decongestants, diuretics, drugs for genito-urinary disorders, drugs for use in parkinsonism and related disorders, drugs for use in rheumatic disorders, hypnotics, minerals and vitamins, lipid lowering drugs and sex hormones. Veterinary drugs may also be suitable for use with the present invention.

Each named drug should be understood to include the neutral form of the drug and pharmaceutically acceptable forms thereof. By “pharmaceutically acceptable forms” thereof is meant any pharmaceutically acceptable derivative or variation, including stereoisomers, stereoisomer mixtures, enantiomers, solvates, hydrates, isomorphs, polymorphs, pseudomorphs, salt forms and prodrugs.

Specific examples of unpleasant-tasting drugs include acetaminophen, albuterol, aminoguanidine hydrochloride, aminophylline, amitriptyline, amoxicillin trihydrate, ampicillin, amlodipine besylate, aspirin, azithromycin, barbiturates, berberine chloride, caffeine, calcium carbonate, calcium pantothenate, cephalosporins, cetirizine, chloramphenicol, chlordiazepoxide, chloroquine, chlorpheniramine, chlorpromazine, cimetidine, ciprofloxacin, clarithromycin, codeine, demerol, dextromethorphan, digitoxin, digoxin, diltiazem hydrochloride, diphenhydramine, diphenylhydantoin, doxazosin mesylate, doxylamine succinate, eletriptan, enoxacin, epinephrine, erythromycin, ethylefrine hydrochloride, etinidine, famotidine, fluconazole, glipizide, guaifenesin, ibuprofen, indeloxazine hydrochloride, lidocaine, lomotil, loratadine, lupitidine, magnesium oxide, meclizine, methacholine, morphine, neostigmine, nifentidine, niperotidine, nizatidine, ofloxacin, paracetamol, pefloxacin, penicillin, phenobarbital, phenothiazine, phenylbutazone, phenylpropanolamine, pipemidic acid, pirbuterol hydrochloride, piroxicam, prednisolone, propranolol hydrochloride, pseudoephedrine, pyridonecarboxylic acid antibacterials, ranitidine, roxatidine, salicylic acid, sertaraline hydrochloride, sildenafil, spironolactone, sulbactam sodium, sulfonamides, sulfotidine, sulpyrine, sultamicillin tosylate, tenidap, terfenadine, theophylline, trimethoprim, tuvatidine, valdecoxib, zaltidine, and zonisamide.

Another class of actives that may benefit from the compositions of the present invention is “low solubility” drugs, meaning that the drug has a minimum aqueous solubility at physiologically relevant pH (e.g., pH 1-8) of about 0.5 mg/mL or less. Cyclodextrins can solubilize low-solubility drugs, enhancing the concentration of dissolved drug when administered to an aqueous environment of use. The, compositions of the present invention are preferred for low-solubility drugs having an aqueous solubility of less than about 0.1 mg/mL, more preferred for low-solubility drugs having an aqueous solubility of less than about 0.05 mg/mL, and even more preferred for low-solubility drugs having an aqueous solubility of less than about 0.01 mg/mL. In general, it may be said that the drug has a dose-to-aqueous solubility ratio greater than about 10 mL, and more typically greater than about 100 mL, where the aqueous solubility (mg/mL) is the minimum value observed in any physiologically relevant aqueous solution (e.g., those with pH values between 1 and 8) including USP simulated gastric and intestinal buffers, and dose is in mg. Thus, a dose-to-aqueous solubility ratio may be calculated by dividing the dose (in mg) by the aqueous solubility (in mg/mL).

Preferred classes of low-solubility drugs include, but are not limited to, antihypertensives, antianxiety agents, anticlotting agents, anticonvulsants, blood glucose-lowering agents, decongestants, antihistamines, antitussives, antineoplastics, beta blockers, anti-inflammatories, antipsychotic agents, cognitive enhancers, cholesterol-reducing agents, anti-atherosclerotic agents, antiobesity agents, autoimmune disorder agents, anti-impotence agents, antibacterial and antifungal agents, hypnotic agents, anti-Parkinsonism agents, anti-Alzheimer's disease agents, antibiotics, anti-depressants, antiviral agents, glycogen phosphorylase inhibitors, and cholesteryl ester transfer protein inhibitors.

Each named drug should be understood to include any pharmaceutically acceptable forms of the drug. Specific examples of antihypertensives include prazosin, nifedipine, amlodipine besylate, trimazosin and doxazosin; specific examples of a blood glucose-lowering agent are glipizide and chlorpropamide; a specific example of an anti-impotence agent is sildenafil and sildenafil citrate; specific examples of antineoplastics include chlorambucil, lomustine and echinomycin; a specific example of an imidazole-type antineoplastic is tubulazole; a specific example of an anti-hypercholesterolemic is atorvastatin calcium; specific examples of anxiolytics include hydroxyzine hydrochloride and doxepin hydrochloride; specific examples of anti-inflammatory agents include betamethasone, prednisolone, aspirin, piroxicam, valdecoxib, carprofen, celecoxib, flurbiprofen and (+)-N-{4-[3-(4-fluorophenoxy)phenoxy]-2-cyclopenten-1-yl}-N-hyroxyurea; a specific example of a barbiturate is phenobarbital; specific examples of antivirals include acyclovir, nelfinavir, and virazole; specific examples of vitamins/nutritional agents include retinol and vitamin E; specific examples of beta blockers include timolol and nadolol; a specific example of an emetic is apomorphine; specific examples of a diuretic include chlorthalidone and spironolactone; a specific example of an anticoagulant is dicumarol; specific examples of cardiotonics include digoxin and digitoxin; specific examples of androgens include 17-methyltestosterone and testosterone; a specific example of a mineral corticoid is desoxycorticosterone; a specific example of a steroidal hypnotic/anesthetic is alfaxalone; specific examples of anabolic agents include fluoxymesterone and methanstenolone; specific examples of antidepression agents include sulpiride, [3,6-dimethyl-2-(2,4,6-trimethyl-phenoxy)-pyridin4-yl]-(1-ethylpropyl)-amine, 3,5-dimethyl-4-(3′-pentoxy)-2-(2′,4′,6′-trimethylphenoxy)pyridine, pyroxidine, fluoxetine, paroxetine, venlafaxine and sertraline; specific examples of antibiotics include carbenicillin indanylsodium, bacampicillin hydrochloride, troleandomycin, doxycyline hyclate, ampicillin and penicillin G; specific examples of anti-infectives include benzalkonium chloride and chlorhexidine; specific examples of coronary vasodilators include nitroglycerin and mioflazine; a specific example of a hypnotic is etomidate; specific examples of carbonic anhydrase inhibitors include acetazolamide and chlorzolamide; specific examples of antifungals include econazole, terconazole, fluconazole, voriconazole, and griseofulvin; a specific example of an antiprotozoal is metronidazole; specific examples of anthelmintic agents include thiabendazole and oxfendazole and morantel; specific examples of antihistamines include astemizole, levocabastine, cetirizine, decarboethoxyloratadine and cinnarizine; specific examples of antipsychotics include ziprasidone, olanzepine, thiothixene hydrochloride, fluspirilene, risperidone and penfluridole; specific examples of gastrointestinal agents include loperamide and cisapride; specific examples of serotonin antagonists include ketanserin and mianserin; a specific example of an anesthetic is lidocaine; a specific example of a hypoglycemic agent is acetohexamide; a specific example of an anti-emetic is dimenhydrinate; a specific example of an antibacterial is cotrimoxazole; a specific example of a dopaminergic agent is L-DOPA; specific examples of anti-Alzheimer's Disease agents are THA and donepezil; a specific example of an anti-ulcer agent/H2 antagonist is famotidine; specific examples of sedative/hypnotic agents include chlordiazepoxide and triazolam; a specific example of a vasodilator is alprostadil; a specific example of a platelet inhibitor is prostacyclin; specific examples of ACE inhibitor/antihypertensive agents include enalaprilic acid and lisinopril; specific examples of tetracycline antibiotics include oxytetracycline and minocycline; specific examples of macrolide antibiotics include erythromycin, clarithromycin, and spiramycin; a specific example of an azalide antibiotic is azithromycin; specific examples of glycogen phosphorylase inhibitors include [R-(R*S*)]-5-chloro-N-[2-hydroxy-3-{methoxymethylamino}-3-oxo-1-(phenylmethyl)propyl]-1H-indole-2-carboxamide and 5-chloro-1H-indole-2-carboxylic acid [(1S)-benzyl-(2R)-hydroxy-3-((3R,4S)-dihydroxy-pyrrolidin-1-yl-)-3-oxypropyl]amide; and specific examples of cholesteryl ester transfer protein (CETP) inhibitors include [2R,4S]4-[(3,5-bis-trifluoromethyl-benzyl)-methoxycarbonyl-amino]-2-ethyl-6-trifluoromethyl-3,4-dihydro-2H-quinoline-1-carboxylic acid ethyl ester, [2R,4S]4-[acetyl-(3,5-bis-trifluoromethyl-benzyl)-amino]-2-ethyl-6-trifluoromethyl-3,4-dihydro-2H-quinoline-1-carboxylic acid isopropyl ester, [2R,4S]4-[(3,5-Bis-trifluoromethyl-benzyl)-methoxycarbonyl-amino]-2-ethyl-6-trifluoromethyl-3,4-dihydro-2H-quinoline-1-carboxylic acid isopropyl ester, (2R)-3-[[3-(4-chloro-3-ethylphenoxy)phenyl][[3-(1,1,2,2-tetrafluoroethoxy)phenyl]methyl]amino]-1,1,1-trifluoro-2-propanol, the drugs disclosed in commonly owned U.S. patent application Ser. Nos. 09/918,127 and 10/066,091, both of which are incorporated herein by reference in their entireties for all purposes, and the drugs disclosed in the following patents and published applications: DE 19741400 A1; DE 19741399 A1; WO 9914215 A1; WO 9914174; DE 19709125 A1; DE 19704244 A1; DE 19704243 A1; EP 818448 A1; WO 9804528 A2; DE 19627431 A1; DE 19627430 A1; DE 19627419 A1; EP 796846 A1; DE 19832159; DE 818197; DE 19741051; WO 9941237 A1; WO 9914204 A1; WO 9835937 A1; JP 11049743; WO 200018721; WO 200018723; WO 200018724; WO 200017164; WO 200017165; WO 200017166; EP 992496; and EP 987251, all of which are hereby incorporated by reference in their entireties for all purposes.

Compositions of the present invention comprising a low-solubility drug and a plurality of particles, the particles comprising cyclodextrin and a water-soluble polymer, can enhance the concentration of the drug in an aqueous use environment. The term “enhance the concentration” means that the particles comprising cyclodextrin and a water-soluble polymer are present in a sufficient amount in the composition so as to improve the concentration of dissolved drug in an aqueous use environment relative to a control composition free from the particles. As used herein, a “use environment” can be either the in vivo environment of the GI tract, subdermal, intranasal, buccal, intrathecal, ocular, intraaural, subcutaneous spaces, vaginal tract, arterial and venous blood vessels, pulmonary tract or intramuscular tissue of an animal, such as a mammal and particularly a human, or the in vitro environment of a test solution, such as phosphate buffered saline (PBS), simulated intestinal buffer without enzymes (SIN), a Model Fasted Duodenal (MFD) solution, or a solution to model the fed state. Concentration enhancement may be determined through either in vitro dissolution tests or through in vivo tests. It has been determined that enhanced drug concentration in in vitro dissolution tests in such in vitro test solutions provide good indicators of in vivo performance and bioavailability. An appropriate PBS solution is an aqueous solution comprising 20 mM sodium phosphate (Na₂HPO₄), 47 mM potassium phosphate (KH₂PO₄), 87 mM NaCl, and 0.2 mM KCl, adjusted to pH 6.5 with NaOH. An appropriate SIN solution is 50 mM KH₂PO₄ adjusted to pH 7.4. An appropriate MFD solution is the same PBS solution wherein additionally is present 7.3 mM sodium taurocholic acid and 1.4 mM of 1-palmitoyl-2-oleyl-sn-glycero-3-phosphocholine. An appropriate solution to model the fed state is the same PBS solution wherein additionally is present 29.2 mM sodium taurocholic acid and 5.6 mM of 1-palmitoyl-2-oleyl-sn-glycero-3-phosphocholine. In particular, a composition of the present invention may be dissolution-tested by adding it to an in vitro test solution and agitating to promote dissolution.

In one aspect, a composition of the present invention, when dosed to an aqueous use environment, provides a maximum drug concentration (MDC) that is at least 1.25-fold the MDC provided by a control composition. In other words, if the MDC provided by the control composition is 100 mg/mL, then a composition of the present invention containing particles comprising cyclodextrin and a water-soluble polymer provides an MDC of at least 125 mg/mL. More preferably, the MDC of drug achieved with the compositions of the present invention are at least 2-fold, even more preferably at least 3-fold, and most preferably at least 5-fold that of the control composition.

The control composition is conventionally drug alone (e.g., typically, the crystalline drug alone in its most thermodynamically stable crystalline form, or in cases where a crystalline form of the drug is unknown, the control may be the amorphous drug alone) or the drug plus a weight of inert diluent equivalent to the weight of the particles used in the test composition. By inert is meant that the diluent is not concentration enhancing.

Alternatively, the compositions of the present invention provide in an aqueous use environment a concentration versus time Area Under the Curve (AUC), for any period of at least 90 minutes between the time of introduction into the use environment and about 270 minutes following introduction to the use environment that is at least 1.25-fold that of the control composition. More preferably, the AUC in the aqueous use environment achieved with the compositions of the present invention are at least 2-fold, more preferably at least 3-fold, and most preferably at least 5-fold that of a control composition.

Alternatively, the compositions of the present invention, when dosed orally to a human or other animal, provide an AUC in drug concentration in the blood plasma or serum that is at least 1.25-fold that observed when an appropriate control composition is dosed. Preferably, the blood AUC is at least about 2-fold, preferably at least about 3-fold, preferably at least about 4-fold, preferably at least about 6-fold, preferably at least about 10-fold, and even more preferably at least about 20-fold that of the control composition. It is noted that such compositions can also be said to have a relative bioavailability of from about 1.25-fold to about 20-fold that of the control composition. Thus, the compositions that, when evaluated, meet either the in vitro or the in vivo, or both, performance criteria are a part of this invention.

Alternatively, the compositions of the present invention, when dosed orally to a human or other animal, provide maximum drug concentration in the blood plasma or serum (Cₘₐₓ) that is at least 1.25-fold that observed when an appropriate control composition is dosed. Preferably, the blood Cₘₐₓ is at least about 2-fold, preferably at least about 3-fold, preferably at least about 4-fold, preferably at least about 6-fold, preferably at least about 10-fold, and even more preferably at least about 20-fold that of the control composition.

A typical in vitro test to evaluate enhanced drug concentration can be conducted by (1) administering with agitation a sufficient quantity of test composition (that is, the low solubility drug and a plurality of particles, the particles comprising cyclodextrin and a water-soluble polymer) in a test medium, such that if all of the drug dissolved, the theoretical concentration of drug would exceed the equilibrium concentration of the drug by a factor of at least 2; (2) in a separate test, adding an appropriate amount of control composition to an equivalent amount of test medium; and (3) determining whether the measured MDC and/or AUC of the test composition in the test medium is at least 1.25-fold that provided by the control composition. In conducting such a dissolution test, the amount of test composition or control composition used is an amount such that if all of the drug dissolved, the drug concentration would be at least 2-fold, preferably at least 10-fold, and most preferably at least 100-fold that of the aqueous solubility (that is, the equilibrium concentration) of the drug.

The concentration of dissolved drug is typically measured as a function of time by sampling the test medium and plotting drug concentration in the test medium vs. time so that the MDC and/or AUC can be ascertained. The MDC is taken to be the maximum value of dissolved drug measured over the duration of the test. The aqueous AUC is calculated by integrating the concentration versus time curve over any 90-minute time period between the time of introduction of the composition into the aqueous use environment (when time equals zero) and 270 minutes following introduction to the use environment (when time equals 270 minutes). Typically, when the composition reaches its MDC rapidly, in say less than about 30 minutes, the time interval used to calculate AUC is from time equals zero to time equals 90 minutes. However, if the AUC of a composition over any 90-minute time period described above meets the criterion of this invention, then the composition formed is considered to be within the scope of this invention.

To avoid drug particulates that would give an erroneous determination, the test solution is either filtered or centrifuged. “Dissolved drug” is typically taken as that material that either passes a 0.45 μm syringe filter or, alternatively, the material that remains in the supernatant following centrifugation. Filtration can be conducted using a 13 mm, 0.45 μm polyvinylidine difluoride syringe filter sold by Scientific Resources under the trademark TITAN®. Centrifugation is typically carried out in a polypropylene microcentrifuge tube by centrifuging at 13,000 G for 60 seconds. Other similar filtration or centrifugation methods can be employed and useful results obtained. For example, using other types of microfilters may yield values somewhat higher or lower (±10-40%) than that obtained with the filter specified above but will still allow identification of preferred dispersions. It is recognized that this definition of “dissolved drug” encompasses not only monomeric solvated drug molecules but also a wide range of species such as drug/cyclodextrin complexes, and other such drug-containing species that are present in the filtrate or supernatant in the specified dissolution test.

Alternatively, the compositions of the present invention, when dosed orally to a human or other animal, results in improved bioavailability or Cₘₐₓ. Relative bioavailability and Cₘₐₓ of drugs in the compositions can be tested in vivo in animals or humans using conventional methods for making such a determination. An in vivo test, such as a crossover study, may be used to determine whether a composition of the present invention provides an enhanced relative bioavailability or Cₘₐₓ compared with a control composition as described above. In an in vivo crossover study a test composition comprising a low-solubility drug and particles, the particles comprising cyclodextrin and a water-soluble polymer, is dosed to half a group of test subjects and, after an appropriate washout period (e.g., one week) the same subjects are dosed with a control composition that consists of an equivalent quantity of crystalline drug as the test composition (but with no cyclodextrin-containing particles present). The other half of the group is dosed with the control composition first, followed by the test composition. The relative bioavailability is measured as the concentration of drug in the blood (serum or plasma) versus time area under the curve (AUC) determined for the test group divided by the AUC in the blood provided by the control composition. Preferably, this test/control ratio is determined for each subject, and then the ratios are averaged over all subjects in the study. In vivo determinations of AUC and Cₘₐₓ can be made by plotting the serum or plasma concentration of drug along the ordinate (y-axis) against time along the abscissa (x-axis). The determination of AUCs and Cₘₐₓ is a well-known procedure and is described, for example, in Welling, “Pharmacokinetics Processes and Mathematics,” ACS Monograph 185 (1986).

### Formation of Compositions

In one embodiment, the invention provides a composition comprising an active and a plurality of particles, the particles comprising cyclodextrin and a water-soluble polymer. The particles are substantially free of the active. The compositions of the present invention may be prepared by first forming the particles comprising the cyclodextrin and polymer and then dry- or wet-mixing the particles with the active. Mixing processes include physical processing as well as wet-granulation and coating processes.

For example, mixing methods include convective mixing, shear mixing, or diffusive mixing. Convective mixing involves moving a relatively large mass of material from one part of a powder bed to another, by means of blades or paddles, revolving screw, or an inversion of the powder bed. Shear mixing occurs when slip planes are formed in the material to be mixed. Diffusive mixing involves an exchange of position by single particles. These mixing processes can be performed using equipment in batch or continuous mode. Tumbling mixers (e.g., twin-shell) are commonly used equipment for batch processing. Continuous mixing can be used to improve composition uniformity.

Milling may also be employed to prepare the compositions of the present invention. Milling conditions are generally chosen that do not alter the physical form of the particles. Conventional mixing and milling processes suitable for use in the present invention are discussed more fully in Lachman, et al., The Theory and Practice of Industrial Pharmacy (3d Ed. 1986).

In addition to the physical mixtures described above, the compositions of the present invention may constitute any device or collection of devices that accomplishes the objective of delivering to the aqueous use environment both the particles and the active. For example, when the active is a pharmaceutical, the composition may be in the form of a dosage form in which the particles and active occupy separate regions within the dosage form. Thus, in the case of oral administration to a mammal, the dosage form may constitute a layered tablet wherein one or more layers comprise the particles and one or more other layers comprise the active.

Although the key ingredients present in the compositions of the present invention are simply the active and the cyclodextrin-containing particles, the inclusion of other excipients in the composition may be useful. These excipients may be included in the particles comprising the cyclodextrin and water-soluble polymer, or may be combined in the compositions with the active.

When the active is a pharmaceutical, conventional formulation excipients may be employed in the compositions of this invention, including those excipients well known in the art (e.g., as described in Remington's Pharmaceutical Sciences (20th ed. 2000)). Generally, excipients such as fillers, disintegrating agents, pigments, binders, lubricants, glidants, flavorants, and so forth may be used for customary purposes and in typical amounts without adversely affecting the properties of the compositions. Chewable tablets may be formulated using conventional tableting excipients such as diluents, swelling agents, anti-tack agents, binders, lubricants, flavorings and sweeteners.

In one embodiment the composition further comprises one or more tastemasking agents. Examples of taste masking agents include sweeteners such as aspartame, compressible sugar, dextrates, lactose, mannitol, maltose, sodium saccharin, sorbitol, and xylitol, and flavors such as banana, grape, vanilla, cherry, eucalyptus oil, menthol, orange, peppermint oil, raspberry, strawberry, and watermelon.

Examples of fillers or diluents include lactose, mannitol, xylitol, dextrose, sucrose, sorbitol, compressible sugar, microcrystalline cellulose, powdered cellulose, starch, pregelatinized starch, dextrates, dextran, dextrin, dextrose, maltodextrin, calcium carbonate, dibasic calcium phosphate, tribasic calcium phosphate, calcium sulfate, magnesium carbonate, magnesium oxide, poloxamers, polyethylene oxide, and hydroxypropyl methyl cellulose.

Examples of surface active agents include sodium lauryl sulfate and polysorbate 80.

Examples of disintegrants include sodium starch glycolate, sodium carboxymethyl cellulose, calcium carboxymethyl cellulose, croscarmellose sodium, crospovidone (polyvinylpyrrolidone), methyl cellulose, microcrystalline cellulose, powdered cellulose, starch, pregelatinized starch, and sodium alginate.

Examples of tablet binders include acacia, alginic acid, carbomer, carboxymethyl cellulose sodium, dextrin, ethylcellulose, gelatin, guar gum, hydrogenatetd vegetable oil, hydroxyethyl cellulose, hydroxypropyl cellulose, hydroxypropyl methyl cellulose, methyl cellulose, liquid glucose, maltodextrin, polymethacrylates, povidone, pregelatinized starch, sodium alginate, starch, sucrose, tragacanth, and zein.

Examples of lubricants include calcium stearate, glyceryl monostearate, glyceryl palmitostearate, hydrogenated vegetable oil, light mineral oil, magnesium stearate, mineral oil, polyethylene glycol, sodium benzoate, sodium lauryl sulfate, sodium stearyl fumarate, stearic acid, talc, and zinc stearate.

Examples of glidants include silicon dioxide, talc and cornstarch.

The composition may be incorporated into a pharmaceutical formulation that may be tasted, including sublingual tablets, chewable tablets, capsules, or unit dose packets, sometimes referred to in the art as “sachets” or “oral powders for constitution” (OPC); syrups; and suspensions. Solid dosage forms, such as chewable tablets for oral administration, are preferred.

One exemplary chewable tablet may be made as follows. First, a pharmaceutical may be combined with particles comprising a cyclodextrin and a water-soluble polymer, a filler such as compressible sugar, microcrystalline cellulose (Avicel PH 101 and Avicel CE), a disintegrant such as Ac-Di-Sol, flavorants, and colorants. These ingredients may be mixed, followed by addition of a lubricant such as magnesium stearate, and then followed by additional mixing. The tablet mixture may be compressed using an F-press and ½″ flat-faced beveled edge tooling, resulting in tablets with a hardness of 7 to 9 kP.

Other features and embodiments of the invention will become apparent from the following examples, which are given for illustration of the invention rather than for limiting its intended scope.

### EXAMPLES

### Example 1

Particles containing β-cyclodextrin and the water-soluble polymer HPMC were prepared using the following procedure. A solution comprising 500 mg β-cyclodextrin and 500 mg hydroxypropyl methyl cellulose (HPMC E3 Prem from Dow) in 15 g deionized water was spray-dried using a “mini spray-drier”. The solution was pumped into a “mini” spray-drying apparatus via a Cole Parmer 74900 series rate-controlling syringe pump at a rate of 24 mL/hr. The solution was atomized through a Spraying Systems Co. two-fluid nozzle, Model No. SU1A using a heated stream of nitrogen at a flow rate of 1 SCFM. The spray solution was sprayed into an 11-cm diameter stainless steel chamber. The heated gas entered the chamber at an inlet temperature of 70° C. and exited at ambient outlet temperature. The resulting particles were collected on filter paper, dried under vacuum, and stored in a desiccator.

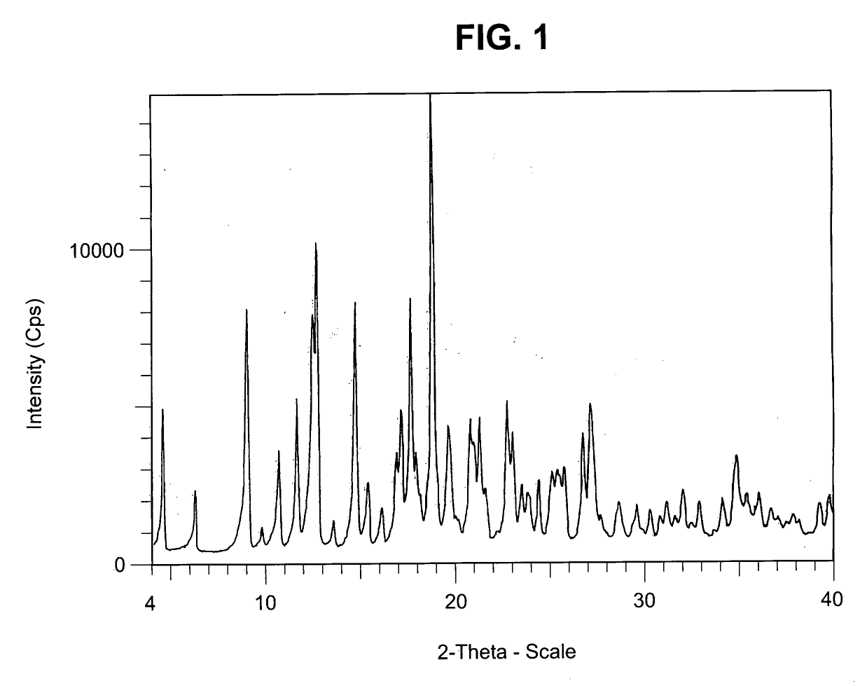

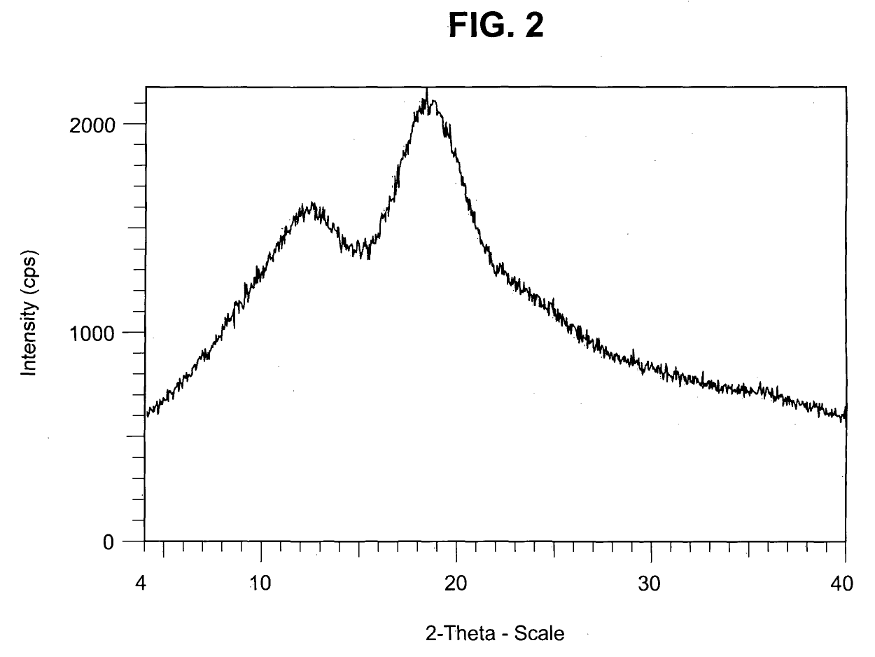

The crystalline β-cyclodextrin used to form the particles and the particles comprising amorphous β-cyclodextrin and HPMC were examined using powder X-ray diffraction with a Bruker AXS D8 Advance diffractometer to determine the amorphous character of the β-cyclodextrin in the particles. Samples (approximately 100 mg) were packed in Lucite sample cups fitted with Si(511) plates as the bottom of the cup to give no background signal. Samples were spun in the j plane at a rate of 30 rpm to minimize crystal orientation effects. The X-ray source (KCua, I=1.54 Å) was operated at a voltage of 45 kV and a current of 40 mA. Data for each sample were collected over a period of 27 minutes in continuous detector scan mode at a scan speed of 1.8 seconds/step and a step size of 0.04°/step. Diffractograms were collected over the 2θ range of 4° to 30°. The results for the crystalline β-cyclodextrin are shown in FIG. 1, showing the characteristic sharp peaks associated with crystalline materials. The results for the amorphous β-cyclodextrin-containing particles are shown in FIG. 2. The 50:50 β-cyclodextrin:HPMC particles exhibited a diffraction pattern showing only an amorphous halo, and no sharp peaks characteristic of crystalline material. These data indicate that the β-cyclodextrin in the particles was amorphous and not crystalline.

### Example 2

Example 1 is repeated except that the water-soluble polymer is hydroxyethyl cellulose (HEC).

### Example 3

Example 1 is repeated except that the water-soluble polymer is hydroxypropyl methyl cellulose acetate succinate (HPMCAS).

### Example 4

Example 1 is repeated except that the water-soluble polymer is poloxamer 407.

### Example 5

Example 1 is repeated except that the water-soluble polymer is sodium carboxymethyl cellulose.

The terms and expressions which have been employed in the foregoing specification are used therein as terms of description and not of limitation, and there is no intention in the use of such terms and expressions of excluding equivalents of the features shown and described or portions thereof, it being recognized that the scope of the invention is defined and limited only by the claims which follow.

We claim:

1. A composition comprising a plurality of particles, each of said particles comprising a cyclodextrin and a water-soluble polymer, wherein said cyclodextrin is in intimate contact with said water-soluble polymer, and wherein at least a major portion of said cyclodextrin is amorphous.

2. The composition of claim 1 further comprising an active, and wherein said particles are substantially free of an active.

3. The composition of claims 1 or 2 wherein said water-soluble polymer is selected from the group consisting of neutral non-cellulosic polymers, ionizable non-cellulosic polymers, neutral cellulosic polymers, and ionizable cellulosic polymers.

4. The composition of claim 3 wherein said water-soluble polymer is selected from the group consisting of polyvinyl alcohols, polyvinyl pyrrolidone, poloxamers, polymethacrylates and polyacrylates, gelatin, hydroxypropyl methyl cellulose, hydroxyethyl cellulose, hydroxypropyl cellulose, hydroxypropyl methyl cellulose acetate succinate, hydroxypropyl methyl cellulose phthalate, carboxymethyl ethyl cellulose, and sodium carboxymethyl cellulose.

5. The composition of claim 4 wherein said water-soluble polymer is hydroxypropyl methyl cellulose.

6. The composition of claims 1 or 2 wherein said cyclodextrin is selected from the group consisting of α-, β- and γ-cyclodextrins.

7. The composition of claim 6 wherein said cyclodextrin is β-cyclodextrin.

8. The composition of claim 2 wherein said active is selected from the group consisting of pharmaceuticals, vitamins, nutriceuticals, agrochemical compounds, nutrients, fertilizers, pesticides, fungicides, botanical extracts, flavoring agents, fruit extracts, spices, cosmetics, coloring agents, and pigments.

9. The composition of claim 8 wherein said active is a pharmaceutical.

10. The composition of claim 9 wherein said pharmaceutical is an unpleasant tasting drug.

11. The composition of claim 9 wherein said pharmaceutical is a low-solubility drug.

12. A process for making a plurality of particles, each of said particles comprising a cyclodextrin and a water-soluble polymer, the process comprising:
(a) forming a solution comprising a cyclodextrin, a water-soluble polymer, and a solvent;
(b) rapidly removing said solvent from said solution to form a solid; and
(c) forming particles from said solid;
wherein at least a major portion of the cyclodextrin in said particles is amorphous.

13. The process of claim 12 wherein step (b) is performed by a process selected from the group consisting of spray-drying, spray-coating, evaporation, lyophilization, and precipitation.

14. The process of claim 13 wherein step (b) is performed by spray drying.

15. The product of the process of any one of claims 12 to 14.
